(19) United States
(12) Patent Application Publication
(10) Pub. No.: US 2009/0123960 A1
(43) Pub. Date: May 14, 2009

(54) Method for Removing Antibiotics From Blood Culture Samples

(75) Inventors: Robert Rosenstein, Ellicott City, MD (US); P. Shawn Beaty, Dallastown, PA (US); Richard Pfeltz, Sparks, MD (US); Matthew Warns, Perry Hall, MD (US)

Correspondence Address: David W. Highet, VP & Chief IP Counsel;Becton, Dickinson and Company, 1 Becton Drive, MC 110, Franklin Lakes, NJ, 07417-1880, US

(73) Assignee: Becton, Dickinson and Company, Franklin Lakes, NJ (US)

(21) Appl. No.: 12/264,647

(22) Filed: Nov. 4, 2008

Related U.S. Application Data
(60) Provisional application No. 60/986,607, filed on Nov. 9, 2007.

(51) Int. Cl. C12Q 1/02; C12N 1/04; C12M 3/00; C12N 1/20

(52) U.S. Cl. 435/29; 435/260; 435/253.6; 435/289.1

(57) ABSTRACT

Methods for removing inhibitors of microbial growth, including antibiotics, from a biological sample suspected of containing one or more microorganisms are provided. The methods include contacting a sample, or a culture growth medium containing the sample, with reversed-phase adsorbent media, which remove the inhibitors of microbial growth, but allow the microorganisms of interest to remain in the sample or culture growth medium.

### FIELD OF THE INVENTION

The presently disclosed subject matter relates to methods for removing inhibitors of microbial growth, including antibiotics, from biological samples, such as a body fluid specimen, and detecting and identifying one or more microorganisms therein.

### BACKGROUND

In many circumstances, the detection and identification of pathogenic microorganisms in biological fluids must be performed in the shortest time possible. For example, in cases of some microbial infections, such as septicemia, the mortality rate remains high in spite of the broad range of antibiotics that are available for treatment. To increase a patient's chance of survival, an antibiotic or a mixture of antibiotics often is administered to the patient before the identity of the infecting microorganism is confirmed. In most cases, however, to achieve the most effective treatment possible, a particular antibiotic therapy regimen based on the identity of the infecting microorganism should be formulated as soon as possible.

Typically, when a biological sample, e.g., a body fluid specimen, is obtained for the purpose of carrying out culturing and subsequent analysis, it is drawn from a subject and incubated in a culture growth medium for a period of time, e.g., several hours. The isolation and rapid characterization of infectious microorganisms can be difficult, however, when the biological sample to be analyzed contains inhibitory agents, such as antibiotics, that can retard or prevent the growth of the microorganism in the culture growth medium. Under such circumstances, the presence of an inhibitory agent in the sample can lead to a false-negative response in the assay. Thus, there is a need in the art for improved methods for removing antibiotics from biological samples. The presently disclosed subject matter addresses, in whole or in part, these and other needs in the art.

### SUMMARY

The presently disclosed subject matter provides methods for removing inhibitors of microbial growth, including antibiotics, from a biological sample suspected of containing one or more microorganisms. The presently disclosed methods include contacting a biological sample, such as a body fluid specimen, including a blood, urine, spinal fluid, or peritoneal fluid specimen, suspected of containing one or more microorganisms with a reversed-phase adsorbent medium, wherein the adsorbent medium selectively removes inhibitors of microorganism growth from the sample. The one or more microorganisms, if present in the sample, are not adsorbed by the adsorbent medium and therefore substantially all of the microorganisms remain in the sample. The ability to remove inhibitors of microorganism growth from the sample, while allowing infecting microorganisms, such as bacteria, to remain and proliferate in the sample or culture growth medium, facilitates the rapid isolation and identification of the infecting microorganism.

Accordingly, in one aspect of the presently disclosed subject matter, a method for selectively removing one or more inhibitors of microorganism growth from a biological sample suspected of containing one or more microorganisms is provided. In some embodiments, the method includes contacting the biological sample suspected of containing one or more microorganisms with a reversed-phase adsorbent medium to selectively remove one or more inhibitors of microorganism growth from the biological sample. Reversed-phase adsorbent media suitable for use with the presently disclosed methods include hydrophobic adsorbent media, including, but not limited to, hydrophobic resins, magnesium-silicate gels, functionalized silica gels, including alkyl-functionalized silica gels, silica gels, and combinations thereof.

In another aspect, a medium for culturing a microorganism is provided, wherein the medium includes a liquid nutrient medium and a reversed-phase adsorbent medium capable of selectively removing an inhibitor of microorganism growth, if present, from the medium.

In yet another aspect, a method for growing and detecting in a body fluid sample an infecting microorganism is provided, the method comprising: providing a culture growth medium comprising a liquid nutrient medium and a reversed-phase adsorbent medium capable of selectively removing an inhibitor of microorganism growth, if present, from the sample or the culture growth medium; inoculating the culture growth medium with the body fluid sample; culturing the inoculated culture growth medium under conditions sufficient to allow growth of the infecting microorganism; and detecting the growth of the infecting microorganism. In some embodiments, the detecting the growth of the infecting microorganism includes detecting metabolic byproducts produced by the infecting microorganism.

In another aspect, a culture growth receptacle for receiving a biological sample suspected of containing one or more inhibitors of microorganism growth is provided, wherein the receptacle includes a culture growth medium and a reversed-phase adsorbent medium capable of selectively removing one or more inhibitors of microorganism growth, if present, in the sample or culture growth medium.

Certain aspects of the presently disclosed subject matter having been stated hereinabove, which are addressed in whole or in part by the presently disclosed subject matter, other aspects will become evident as the description proceeds when taken in connection with the accompanying Examples as best described herein below.

### DETAILED DESCRIPTION

The presently disclosed subject matter now will be described more fully hereinafter, with reference to the accompanying Examples, in which some, but not all embodiments of the presently disclosed subject matter are illustrated. Many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. Although specific terms are employed herein, they are used in a generic and descriptive sense only and not for purposes of limitation.

The terms “a,” “an,” and “the” refer to “one or more” when used in this application, including the claims. Thus, for example, reference to “a sample” includes a plurality of samples, unless the context clearly is to the contrary (e.g., a plurality of samples), and so forth.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

I. Method for Selectively Removing One or More Inhibitors of Microorganism Growth from a Culture Growth Medium or Biological Sample

A. General Considerations

The presence of microorganisms, such as bacteria, in a body fluid specimen can be detected by inoculating a volume of the specimen in a culture growth medium and observing the appearance of turbidity or by detecting metabolic byproducts produced by the microorganism, each of which is an indication of microorganism growth. The detection and identification of microorganisms in a biological sample drawn from a subject who has been administered one or more antimicrobial drugs, however, can be difficult. Under such circumstances, the one or more antimicrobial drugs can be transferred along with the microorganism into the culture growth medium. The presence of one or more antimicrobial drugs in the culture growth medium can inhibit the growth of or kill the microorganism, thus interfering with isolation and identification of the microorganism. Under some circumstances, the presence of the antimicrobial agent in the culture growth medium can delay isolation of the microorganism for more than two weeks, which can be detrimental to the diagnosis and treatment of the microbial infection. Additionally, in blood samples, for example, the isolation of the microorganism of interest also can require excess periods of incubation because of inhibitors of microorganism growth contained in serum, plasma, or lysed erythrocytes. Similar problems exist despite the type of body fluid being examined, whether the fluid is urine, spinal fluid, abscess exudates, serum, peritoneal fluid, and the like.

In previously disclosed methods, removal of antibiotics from biological samples can be accomplished by using resins. Typically, these resins are polystyrene based and are cross-linked with divinyl benzene. Such resins can be charged resins, e.g., ion exchange resins, or uncharged resins. If the substance to be bound is either negatively or positively charged, an ion exchange resin can be used. A variety of commercial resins can be used for removing antibiotics from biological samples, including DOWEX™ ion exchange resins (The Dow Chemical Co., Midland, Mich.) and AMBERLITE™ ion exchange and polymeric resins (Rohm and Haas Company, Philadelphia, Pa.).

Resin-based methods for removing antibiotics from a biological sample typically involve a two-step process: a sample, e.g., a blood sample, to be eventually cultured for bacteria is first passed over a bed of suitably prepared resin, and the filtrate, presumably with the antibiotics and other interfering substances removed by the resin, is then added to liquid growth media for biological amplification of any bacteria present in the sample. This method is the subject of U.S. Pat. No. 4,174,277 to Melnick et al. (hereinafter “the '277 patent”), which is incorporated by reference in its entirety. In this method, the resins are either ion-exchange resins or uncharged resins, which first must be treated, e.g., fluidized, with a non-ionic detergent. If such resins are not treated with a non-ionic detergent, any bacteria that might be present in the sample can be removed as well as the unwanted antibiotics that also might be present in the sample.

Another resin-based method for removing antibiotics from biological samples is disclosed in U.S. Pat. Nos. 4,632,902 and 5,624,814 to Waters et al. (hereinafter “the '902 patent” and “the '814 patent,” respectively), each of which is incorporated herein by reference in its entirety. The method disclosed in the '902 patent and the '814 patent includes adding the prepared resins, i.e., resins fluidized with non-ionic detergent, directly to the growth media so that the removal of antibiotics occurs continuously during the growth phase and does not require the two-step process disclosed in the '277 patent to Melnick et al. The method disclosed in the '902 and the '814 patents to Waters et al. currently is considered the standard resin-based antibiotic removal method and is widely used.

Another approach to removing antibiotics from biological samples includes disposing activated charcoal in the growth media, see, e.g., U.S. Pat. No. 5,314,855 to Thorpe et al., or passing the biological fluid over a fibrous material formed from activated carbon fibers, see, e.g., U.S. Pat. No. 5,897,782 to Chatelin et al. The charcoal method, however, generally is recognized as not being as robust as the resin-based methods discussed hereinabove.

B. Method for Selectively Removing One or More Inhibitors of Microorganism Growth

In some embodiments, the presently disclosed subject matter provides a method for removing inhibitors of microorganism growth, such as antibiotics and other interfering substances from a biological sample, including a blood sample, during culturing of the sample in liquid media by using the principle of reversed-phase chromatography. Standard chromatography methods, such as high performance liquid chromatography and thin layer chromatography, include normal-phase chromatography and reversed-phase chromatography. In normal-phase chromatography, two phases are established, an immobile, i.e., stationary, polar (hydrophilic) phase and a mobile non-polar (hydrophobic) phase. In reversed-phase chromatography, two phases also are established, but the phases are reversed relative to normal-phase chromatography. That is, reversed-phase chromatography includes a mobile polar (hydrophilic) phase and an immobile, i.e., stationary, non-polar (hydrophobic) phase.

Antibiotics, or other inhibitors of microorganism growth, can partition between the mobile and stationary phases of a reversed-phase chromatographic separation process. If the target molecule, e.g., an antibiotic, is more hydrophobic than hydrophilic, the target molecule will predominately associate with the hydrophobic stationary phase. In some embodiments of the presently disclosed subject matter, the stationary phase materials are less polar, i.e., more hydrophobic, than the water-based media used for bacterial growth.

Accordingly, the presently disclosed subject matter provides a method for selectively removing one or more inhibitors of microorganism growth from a biological sample suspected of containing one or more microorganisms, the method including: (a) providing a reversed-phase adsorbent medium; and (b) contacting the biological sample suspected of containing one or more microorganisms with the reversed-phase adsorbent medium to selectively remove one or more inhibitors of microorganism growth from the biological sample.

As used herein, the term “selectively removes” is intended to include adsorbing, isolating, extracting, or otherwise removing one or more inhibitors of microorganism growth from a biological sample while leaving substantially all of the microorganisms, if present, in the sample. By “substantially all” is meant, in some embodiments more than 99% of the microorganisms remain in the sample; in some embodiments, more than 98%, in some embodiments, more than 97%; in some embodiments more than 95%; in some embodiments, more than 90%; in some embodiments, more than 85%; in some embodiments, more than 80%; and in some embodiments, more than 75% of the microorganisms remain in the sample.

The reversed-phase adsorbent medium can include a hydrophobic resin, a magnesium-silicate gel, an alkyl-functionalized silica gel, a silica gel, and combinations thereof. More particularly, the reversed-phase adsorbent medium can include hydrophobic reversed-phase chromatographic materials, such as octyl-functionalized (C8) silica gel; octadecyl-functionalized (C18) silica gel; hydrophobic resins, such as CALBIOSORB™ (EMD Chemicals, Inc., San Diego, Calif.); magnesia-silica gels, such as FLORISIL® (U.S. Silica Company, Berkeley Springs, W.V.); and silica gels, such as Silica Gel A (Riedel-de Haen) (Sigma-Aldrich, St. Louis, Mo.). Such adsorbent materials also are referred to herein as a “reversed-phase adsorbent medium,” “adsorbers” or “inhibitor removers.” Other reversed-phase adsorbent media, including, but not limited to, butyldimethyl (C-4) silica gel, phenyl-bonded silica gel, and cyanopropyl-bonded silica gel, also could be used in the presently disclosed methods. Not all hydrophobic adsorbent media, however, are suitable for use with the presently disclosed methods. As disclosed herein, it has been surprisingly found that the best adsorbers have some hydrophilic, or polar, characteristics.

In some embodiments of the presently disclosed methods, the microorganism is a bacterium and the one or more inhibitors of microorganism growth is an antibiotic. More generally, as used herein, the phrase “inhibitors of microorganism growth” includes antimicrobial agents, such as one or more antibiotic. Accordingly, the terms “inhibitors of microorganism growth,” “antimicrobial agents,” “inhibitory agents,” or “inhibitors” are used herein to denote any agent intended for preventing the proliferation, e.g., growth, of microbes or pathogenic microorganisms, including, but not limited to, an antibiotic, an antifungal, an antiviral, and an antiseptic.

Representative classes of antibiotics include, but are not limited to, a macrolide, a cephalosporin, a tetracycline, a quinoline, including fluoroquinolones, an aminoglycoside, a glycopeptide, a carbacephem, a carbapenem, a monobactam, a penicillin, a sulfonamide, a polypeptide, and combinations thereof. Representative antibiotics belonging to these general classes of antibiotics are provided herein below.

An aminoglycoside, including, but not limited to amikacin, gentamicin, kanamycin, neomycin, netilmicin, streptomycin, tobramycin, and paromomycin; an ansamycin, including, but not limited to, geldanamycin and herbimycin. A carbacephem, including, but not limited to, loracarbef. A carbapenem, including, but not limited to, ertapenem, doripenem, imipenem/cilastatin, and meropenem. First generation cephalosporins, including, but not limited to, cefadroxil, cefazolin, cefalotin, and cephalexin. Second generation cephalosporins, including, but not limited to, cefaclor, cefamandole, cefoxitin, cefprozil, and cefuroxime. Third generation cephalosporins, including, but not limited to, cefixime, cefdinir, cefditoren, cefoperazone, cefotaxime, cefpodoxime, ceftazidime, ceftibuten, ceftizoxime, ceftriaxone, and cefdinir. Fourth generation cephalosporins, including, but not limited to cefepime. Glycopeptides, including, but not limited to, teicoplanin and vancomycin. Macrolides, including, but not limited to azithromycin, clarithromycin, dirithromycin, erythromycin, roxithromycin, telithromycin, and spectinomycin. Monobactams, including but not limited to aztreonam. Penicillins, including, but not limited to, amoxicillin, ampicillin, azlocillin, carbenicillin, cloxacillin, dicloxacillin, flucloxacillin, mezlocillin, nafcillin, penicillin, piperacillin, and ticarcillin. Polypeptides, including, but not limited to, bacitracin, colistin, and polymyxin B. Quinolones, including, but not limited to, ciprofloxacin, enoxacin, gatifloxacin, levofloxacin, lomefloxacin, moxifloxacin, norfloxacin, ofloxacin, and trovafloxacin. Sulfonamides, including, but not limited to, mafenide, prontosil, sulfacetamide, sulfamethizole, sulfanilamide, sulfasalazine, sulfisoxazole, trimethoprim, and trimethoprim-sulfamethoxazole (co-trimoxazole (TMP-SMX). Tetracyclines, including, but not limited to demeclocycline, doxycycline, minocycline, oxytetracycline, and tetracycline. And other antibiotics, including, but not limited to, arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin, and tinidazole.

As provided in more detail herein below, the biological sample can include a body fluid sample. In some embodiments, the body fluid sample can include a blood specimen, a urine specimen, a spinal fluid specimen, or a peritoneal fluid specimen.

C. Medium for Culturing a Microorganism

In some embodiments, the presently disclosed subject matter provides a medium for culturing a microorganism, the medium comprising a liquid nutrient medium and a reversed-phase adsorbent medium capable of selectively removing an inhibitor of microorganism growth, if present, from the medium. In some embodiments, the liquid nutrient medium is a hypertonic medium. The reversed-phase adsorbent medium can include a hydrophobic resin, a magnesium silicate gel, a silica gel, an alkyl-functionalized silica gel, and combinations thereof, and described hereinabove.

Accordingly, in some embodiments of the presently disclosed methods, the reversed-phase adsorbent medium is disposed in a growth medium. Any growth medium suitable for use in cultivating one or more microorganisms of interest can be used with the presently disclosed methods. The cultivating of one or more microorganisms in a growth medium is, of course, limited to microorganisms that will grow in that particular type of medium. Representative growth media include, but are not limited commercially available growth media, including BACTEC™ growth media (BD Diagnostic Systems, Sparks, Md.).

In some embodiments, a general growth medium, such as a soybean-casein digest broth can be used. Such broths can contain, for example, a digest, e.g., a pancreatic digest, of casein and a digest, e.g., a papaic digest, of soybean meal in an aqueous solution. One of ordinary skill in the art would recognize that such culture broths also can include other ingredients, including, but not limited to, yeast extract, animal tissue digest, sucrose, dextrose, fructose, arginine, hemin, menadione, thiols, pyridoxal HCl (Vitamin B₆), sodium carbonate, sodium bicarbonate, sodium citrate, potassium phosphate, sodium polyanetholsulfonate (SPS), and combinations thereof. In some embodiments, the growth medium includes a growth factor, including, but not limited to, NAD or factor V. Such growth factors can be used as a supplement to improve the recovery and detection of fastidious microorganisms, such as Haemophilus species.

Representative aerobic and anaerobic microorganisms suitable for use with the presently disclosed culture media include, but are not limited to, Acinetobacter baumannii; Acinetobacter lwoffli; Alcaligenes faecalis; Bacteroides fragilis; Bacteroides vulgatus; Candida albicans; Candida (Torulopsis) glabrata; Clostridium histolycum; Clostridium perfringens; Corynebacterium J-K; Cryptococcus neoformans; Enterobacter cloacae; Escherichia coli; Haemophilus influenzae; Haemophilus parainfluenzae; Klebsiella pneumoniae; Neisseria gonorrhoeae; Neisseria meningitidis; Proteus mirabilis; Providencia stuarti; Pseudomonas aeruginosa; Salmonella typhimurium; Serratia marcescens; Staphylococcus aureus; Staphylococcus epidermidis; Stenotrophomonas (Xanthomonas) maltophilia; Streptococcus pneumoniae; Streptococcus pyogenes; Group A Streptococcus; and Group D Streptococcus.

In some embodiments, the reversed-phase adsorbent medium is disposed in a growth bottle. The reversed-phase adsorbent media can be disposed in the bottle either before or after the sample or the culture growth medium is disposed in the bottle. Accordingly, a culture growth bottle can include a culture growth medium, the reversed-phase adsorbent media, or a mixture of the culture growth medium and the reversed-phase adsorbent media. In some embodiments, the presently disclosed subject matter includes a culture growth receptacle for receiving a biological sample suspected of containing one or more inhibitors of microorganism growth, wherein the receptacle includes a culture growth medium and a reversed-phase adsorbent medium capable of selectively removing one or more inhibitors of microorganism growth, if present, in the sample or culture growth medium. The culture growth receptacle can include a bottle, a vial, a tube, or any suitable container for receiving a biological sample.

When disposed in a culture growth medium or in a receptacle containing a volume of a culture growth medium, the reversed-phase adsorbent medium can comprise, in some embodiments, between about 5% to about 25% weight/volume of the growth medium; in some embodiments, between about 10% to about 20% weight/volume of the growth medium; in some embodiments, between about 12.5% to about 17.5% weight/volume of the growth medium; in some embodiments, about 15.0% weight/volume of the growth medium; and, in some embodiments, about 10% weight/volume of the growth medium.

In some embodiments, the sample volume disposed in the culture growth receptacle has a range from about 5 mL to about 7 mL. In some embodiments, the growth vial or bottle can be formulated to accommodate a sample volume up to about 10 mL of blood. In yet other embodiments, the growth vial or bottle can be formulated to accommodate a sample volume of up to about 25 mL or more. The addition of such large sample volumes can result in higher detection rates and earlier times of detection. Smaller vials, e.g., vials that can accommodate blood specimens generally less than about 3 mL in volume, also can be used for samples obtained from, for example, pediatric patients.

D. Method for Growing and Detecting in a Body Fluid Sample an Infecting Microorganism

In some embodiments, the presently disclosed subject matter provides a method for growing and detecting in a body fluid sample an infecting microorganism. In some embodiments, the method includes: (a) providing a culture growth medium comprising a liquid nutrient medium and a reversed-phase adsorbent medium capable of selectively removing an inhibitor of microorganism growth, if present, from the sample or the culture growth medium; (b) inoculating the culture growth medium with the body fluid sample; (c) culturing the inoculated culture growth medium under conditions sufficient to allow growth of the infecting microorganism; and (d) detecting the growth of the infecting microorganism. In some embodiments, the liquid nutrient medium is a hypertonic medium. The reversed-phase adsorbent medium can include a hydrophobic resin, a magnesium silicate gel, a silica gel, an alkyl-functionalized silica gel, and combinations thereof, as described hereinabove.

The presently disclosed methods of adsorbing or removing one or more antimicrobial agents from a biological sample can be carried out immediately following the drawing of the biological sample from a subject and before the sample is cultured. Such methods can reduce the occurrence of interferences and promote the growth of the microorganisms, if present, in the sample to be analyzed.

During the inoculating and culturing steps described hereinabove, the sample can be disposed in a culture growth bottle containing the reversed-phase adsorbent media. The bottle can be continuously agitated during the growth phase, thereby creating and re-creating a polar mobile phase in continuous contact with the reversed-phase adsorbent medium. Without wishing to be bound to any particular theory, it is believed that the driving force in the binding of the inhibitor of microorganism growth, e.g., an antibiotic, to the reversed-phase adsorbent medium is the decrease in the area of the non-polar segment of the antibiotic molecule exposed to the growth medium or solvent. This hydrophobic effect is dominated by a decrease in free energy from entropy associated with the minimization of the ordered molecule-polar solvent interface. This mechanism can be achieved because the target molecules, i.e., one or more inhibitors of microorganism growth, have enough hydrophobic character to be preferentially associated with the hydrophobic, non-polar reversed-phase medium instead of the aqueous (polar) environment of the growth medium. Again, without wishing to be bound to any one particular theory, the efficacy of the presently disclosed methods can be attributed to the “solvent,” e.g., a water-based liquid bacterial culture growth medium, being highly polar (hydrophilic).

In some embodiments, the detecting the growth of the infecting microorganism includes detecting metabolic byproducts produced by the infecting microorganism. In such embodiments, the culture growth broth can be contained in a culture growth vial, also referred to herein as a culture growth bottle, e.g., a BACTEC™ growth bottle (BD Diagnostic Systems, Sparks, Md.), which can be enriched with CO₂ and, in some embodiments, enriched with a combination of CO₂ and N₂. Such culture growth vials or bottles can be used in conjunction with, for example, a fluorescence instrument to culture and detect microorganisms, such as bacteria and yeast, in a biological sample, including blood. If microorganisms are present in the sample inoculated in the growth medium, CO₂ will be produced when the microorganisms metabolize the ingredients in the growth medium. In such embodiments, the vial can include a chemical sensor that can measure the rate and amount of CO₂ produced by the growth of microorganisms.

In representative embodiments, the sample is incubated for a period of time in the growth medium, e.g., about 72 hours or less, and the sensor is monitored periodically to detect an increase in fluorescence, which is proportional to the amount of CO₂ present in the vial. A positive response as determined by the rate and amount of CO₂ increase in the vial indicates the presence of viable microorganisms in the sample.

In some embodiments of the presently disclosed methods, the biological sample includes a body fluid specimen. The body fluid specimen can include blood, urine, spinal fluid, or peritoneal fluid. More particularly, as used herein, the term “sample” includes any liquid or fluid sample, including a sample derived from a biological source, and can be referred to herein as a biological fluid. The term “biological fluid,” as used herein, includes any fluid containing or suspected of containing living matter, such as a physiological fluid, including whole blood or whole blood components, such as red blood cells, white blood cells, platelets, serum and plasma; ascites; urine; saliva; sweat; milk; synovial fluid; peritoneal fluid; amniotic fluid; percerebrospinal fluid; lymph fluid; lung embolism; cerebrospinal fluid; pericardial fluid; cervicovaginal samples; tissue extracts; cell extracts; and other constituents of the body that are suspected of containing the one or more microorganisms of interest. In addition to physiological fluids, other liquid samples, such as water, food products and the like, for the performance of environmental or food production assays are suitable for use with the presently disclosed subject matter. A solid material suspected of containing the analyte also can be used as the test sample. In some instances it can be beneficial to modify a solid test sample to form a liquid medium for use in the assay.

In some embodiments, the sample can be pre-treated prior to use, such as preparing plasma from blood, diluting viscous fluids, or the like. Such methods of treatment can involve filtration, distillation, concentration, inactivation of interfering compounds, and the addition of reagents.

The sample can be any sample obtained from a subject. The term “subject” refers to an organism, tissue, or cell from which a sample can be obtained. A subject can include a human subject for medical purposes, such as diagnosis and/or treatment of a condition or disease, or an animal subject for medical, veterinary purposes, or developmental purposes. A subject also can include sample material from tissue culture, cell culture, organ replication, stem cell production and the like. Suitable animal subjects include mammals and avians. The term “avian” as used herein includes, but is not limited to, chickens, ducks, geese, quail, turkeys, and pheasants. The term “mammal” as used herein includes, but is not limited to, primates, e.g, humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. Preferably, the subject is a mammal or a mammalian cell. More preferably, the subject is a human or a human cell. Human subjects include, but are not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. A subject also can refer to cells or collections of cells in laboratory or bioprocessing culture in tests for viability, differentiation, marker production, expression, and the like.

The presently disclosed methods can be used to diagnose, for the prognosis, or the monitoring of a disease state or condition. As used herein, the term “diagnosis” refers to a predictive process in which the presence, absence, severity or course of treatment of a disease, disorder or other medical condition is assessed. For purposes herein, diagnosis also includes predictive processes for determining the outcome resulting from a treatment. Likewise, the term “diagnosing,” refers to the determination of whether a sample specimen exhibits one or more characteristics of a condition or disease. The term “diagnosing” includes establishing the presence or absence of one or more microorganisms, or establishing, or otherwise determining one or more characteristics of a condition or disease, including type, grade, stage, or similar conditions. As used herein, the term “diagnosing” can include distinguishing one form of a disease from another. The term “diagnosing” encompasses the initial diagnosis or detection, prognosis, and monitoring of a condition or disease.

The term “prognosis,” and derivations thereof, refers to the determination or prediction of the course of a disease or condition. The course of a disease or condition can be determined, for example, based on life expectancy or quality of life. “Prognosis” includes the determination of the time course of a disease or condition, with or without a treatment or treatments. In the instance where treatment(s) are contemplated, the prognosis includes determining the efficacy of a treatment for a disease or condition.

The term “monitoring,” such as in “monitoring the course of a disease or condition,” refers to the ongoing diagnosis of samples obtained from a subject having or suspected of having a disease or condition.

As used herein, the term “risk” refers to a predictive process in which the probability of a particular outcome is assessed.

The term “marker” refers to a molecule, such as a protein, including an antigen, that when detected in a sample is characteristic of or indicates the presence of a disease or condition.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

### General Experimental Protocol

In some embodiments of the presently disclosed subject matter, Staphylococcus aureus (S. aureus) ATCC 29213 (American Type Culture Collection (ATCC), Manassas, Va.) was used as the test organism. S. aureus ATCC 29213 was grown overnight from −70° C. freezer stocks streaked onto BBL™ TRYPTICASE™ Soy Agar with 5% sheep blood (TSA II™) (BD Diagnostic Systems, Sparks, Md.) the day before culture inoculation. Plates were inverted and incubated at 35° C. On the day of a set of culture inoculations, growth was taken from the plate with a sterile, cotton-tipped swab and suspended in sterile saline to 1.0 McFarland's turbidity standard as determined with a CRYSTAL SPEC™ hand-held nephelometer (BD Diagnostic Systems, Sparks, Md.). This suspension was diluted by 1×10⁻⁶ in Standard Aerobic BACTEC™ medium (BD Diagnostic Systems, Sparks, Md.) and each culture bottle was injected with 0.1 mL of this dilution using a 1-cc syringe/27 gauge needle, expected to provide between about 10 colony forming units (CFU)/bottle to about 100 CFU/bottle. Antimicrobial agents were added to culture bottles with syringes just prior to inoculation.

Antimicrobial stocks were prepared ahead of time and stored at −70° C. in accordance with Clinical and Laboratory Standards Institute (CLSI, Wayne, Pa.) guidelines. The titer of each inoculum prepared was confirmed with a plate count made by plating 0.1 mL by syringe on BBL™ TRYPTICASE™ Soy Agar with 5% sheep blood (TSA II™) blood agar (BD Diagnostic Systems, Sparks, Md.) that was then inverted and set at 35° C. to incubate. Colonies were counted the following day. For this particular example, culturing in BACTEC™ bottles (BD Diagnostic Systems, Sparks, Md.) was done without added blood. The total fluid volume in each bottle was 25 mL.

### Example 2

### Medium Preparation for Inhibitor Removers

For each inhibitor remover, 2 grams were weighed out into each of 20 weigh boats then added to sensor bottles using the end segment of a 60-cc syringe with the threads bored out. The narrow end of a wooden-handled cotton swab was used to push resins through the syringe segment. Different weigh boats were used for the different inhibitor removers. Seven liters (enough for 280×25 mL) of BBL™ TRYPTICASE™ Soy Broth (TSB) (BD Diagnostic Systems, Sparks, Md.) were prepared at a concentration of about 30 g/L. A Wheaton UNISPENSE® liquid dispenser (Wheaton Science Products, Millville, N.J.) was used to fill the sensor bottles containing inhibitor adsorbers (removers) with 25-mL TSB per bottle. The bottles were then capped. Four trays of bottles, one per inhibitor remover type, were autoclaved for 20 minutes at 121° C. on liquid cycle.

### Example 3

### Representative Reversed-Phase Adsorbent Media

In some embodiments, the reversed-phase adsorbent medium is a hydrophobic resin, e.g., CALBIOSORB™ (EMD Biosciences, San Diego, Calif. (Catalog No. 206550)). In some embodiments, the reversed-phase adsorbent medium is a magnesia-silica gel MgO 3.75 SiO₂(x)H₂O, e.g., FLORISIL® (EMD Biosciences (Catalog No. FX0284-1)). In some embodiments, the reversed-phase adsorbent medium is an octyl-functionalized silica gel [SiO]ₓ—[CH₂(CH₂)₆—CH₃]_{y}, e.g., C8-Silica Gel (Sigma-Aldrich, St. Louis, Mo. (Catalog No. 385441)). In some embodiments, the reversed-phase adsorbent medium is an octadecyl-functionalized silica gel [SiO]ₓ—[CH₂(CH₂)₁₆—CH₃]_{y}, e.g., C18-Silica Gel (Sigma-Aldrich (Catalog No. 377635)). In some embodiments, the reversed-phase adsorbent medium is a silica gel (O═Si═O), e.g., Silica Gel A (Sigma-Aldrich (Riedel-de Haen, Catalog No. 10081)).

### Example 4

### Preparation of Representative Reversed-Phase Adsorbent Medium

CALBIOSORB™ was washed 5 times with distilled water at 60° C. Excess liquid was poured off and the CALBIOSORB™ was stored at 4° C. until used. FLORISIL® was washed 5 times with distilled water at 60° C. Excess liquid was poured off and the FLORISIL® was stored at 4° C. until used. C8-Silica Gel was washed with anhydrous ethanol for 30 minutes. After the ethanol was poured off, distilled water at 60° C. was used to wash the C8-Silica Gel five times. Excess water was poured off and the C8-Silica Gel was stored at 4° C. until used. C18-Silica Gel was prepared using the same process as used for the C8-Silica Gel. Silica Gel A (washed) was washed five times with distilled water at 60° C. Silica Gel (converted) was initially washed with 1N NaOH instead of water, followed by five washes with distilled water (at 60° C.) to remove residual NaOH.

### Example 5

### Preparation of Representative Antibiotics

Azithromycin (AZM), a broad-spectrum antibiotic related to erythromycin, used at 8.57 micrograms per mL in the bottle. Ceftriaxone sodium (CTR), a cephalosporin, used at 2.86 micrograms per mL in the bottle. Doxycycline hyclate (DOX), a tetracycline, used at 1.14 micrograms per mL in the bottle. Gatifloxacin (GAT), a broad-spectrum fluoroquinolone, used at 0.17 micrograms per mL in the bottle. Gentamicin sulfate (GEN), an aminoglycoside, used at 2.29 micrograms per mL in the bottle. Teicoplanin (TEC), a glycopeptide antibiotic related to vancomycin, used at 1.14 micrograms per mL in the bottle.

### Example 6

The results from the detection of S. aureus in culture growth media containing selected antibiotics, wherein the antibiotics are selectively removed by representative reversed-phase adsorbent media, are presented in Table 1.

As shown in Table 1, several of the presently disclosed reversed-phase adsorbent media were effective in removing one or more of the antibiotics from the culture growth medium, while allowing the added S. aureus cells to grow in the medium. FLORISIL® (MgO 3.75 SiO₂(x)H₂O) was found to be the most effective adsorbent medium and compared well to commercially available culture growth media that incorporate resins. CALBIOSORB™ was the next most effective adsorbent medium. Washed silica Gel A was somewhat effective against AZM, but not CTR. Converted silica gel A was effective against CTR but not AZM. C8-silica gel is more effective than C18-silica gel.

TABLE 1
Detection of S. aureus in Culture growth Media
Containing Selected Antibiotics

| Culture Media +/− | Time To Detection (hrs) | | | | | | |
|---|---|---|---|---|---|---|---|
| Adsorbent Media | none | AZM | CTR | DOX | GAT | GEN | TEC |
| Standard Aerobic^{†} | | | | | | | |
| Mean | 11.04 | none | none | none | none | none | none |
| SD | 0.38 | none | none | none | none | none | none |
| # Detections/# Cultures | 17/17 | 0/11 | 0/14 | 0/8 | 0/8 | 0/8 | 0/8 |
| Detection Rate (%) | 100% | 0% | 0% | 0% | 0% | 0% | 0% |
| Plus Aerobic^{‡} | | | | | | | |
| Mean | 12.87 | 13.95 | 13.32 | 13.50 | 12.56 | 12.97 | 13.59 |
| SD | 0.47 | 0.24 | 0.58 | 0.40 | 0.09 | 0.25 | 0.50 |
| # Detections/# Cultures | 15/15 | 12/12 | 15/15 | 6/6 | 6/6 | 6/6 | 6/6 |
| Detection Rate (%) | 100% | 100% | 100% | 100% | 100% | 100% | 100% |
| CALBIOSORB ™ | | | | | | | |
| Mean | 12.50 | 14.31 | none | 35.47 | 14.75 | 20.94 | 13.53 |
| SD | 0.00 | 0.19 | none | 2.18 | 0.69 | 1.52 | 0.61 |
| # Detections/# Cultures | 6/6 | 6/6 | 0/6 | 6/6 | 6/6 | 6/6 | 6/6 |
| Detection Rate (%) | 100% | 100% | 0% | 100% | 100% | 100% | 100% |
| FLORISIL ® | | | | | | | |
| Mean | 12.59 | 35.22 | 67.64 | 22.51 | 13.50 | 15.59 | 45.69 |
| SD | 0.61 | 2.87 | 35.54 | 2.07 | 0.38 | 1.88 | 5.43 |
| # Detections/# Cultures | 6/6 | 6/6 | 2/6 | 6/6 | 6/6 | 6/6 | 6/6 |
| Detection Rate (%) | 100% | 100% | 33% | 100% | 100% | 100% | 100% |
| C8 Silica Gel | | | | | | | |
| Mean | 13.75 | 15.89 | none | none | 50.10 | 79.53 | 14.92 |
| SD | 0.69 | 0.61 | none | none | 24.86 | 48.18 | 1.40 |
| # Detections/# Cultures | 6/6 | 6/6 | 0/6 | 0/6 | 6/6 | 3/6 | 6/6 |
| Detection Rate (%) | 100% | 100% | 0% | 0% | 100% | 50% | 100% |
| C18 Silica Gel | | | | | | | |
| Mean | 12.17 | 56.84 | none | none | 104.70 | none | none |
| SD | 0.19 | 6.62 | none | none | none | none | none |
| # Detections/# Cultures | 6/6 | 6/6 | 0/6 | 0/6 | 1/6 | 0/6 | 0/6 |
| Detection Rate (%) | 100% | 100% | 0% | 0% | 17% | 0% | 0% |
| Silica Gel A washed | | | | | | | |
| Mean | 11.34 | 104.18 | none | | | | |
| SD | 0.28 | 9.76 | none | | | | |
| # Detections/# Cultures | 12/12 | 4/9 | 0/7 | | | | |
| Detection Rate (%) | 100% | 44% | 0% | | | | |
| Silica Gel A converted | | | | | | | |
| Mean | 30.22 | none | 31.78 | | | | |
| SD | 8.07 | none | 6.24 | | | | |
| # Detections/# Cultures | 13/14 | 0/6 | 9/11 | | | | |
| Detection Rate (%) | 93% | 0% | 82% | | | | |

^{†}Standard Aerobic refers to BACTEC ™ Standard Aerobic Culture growth medium (BD Diagnostic Systems, Sparks, Maryland);
^{‡}Plus Aerobic refers to BACTEC ™ Plus Aerobic Culture growth medium, which incorporates polystyrene-based resins.

All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for selectively removing one or more inhibitors of microorganism growth from a biological sample suspected of containing one or more microorganisms, the method comprising:
(a) providing a reversed-phase adsorbent medium;
(b) contacting the biological sample suspected of containing one or more microorganisms with the reversed-phase adsorbent medium to selectively remove one or more inhibitors of microorganism growth from the biological sample.

2. The method of claim 1, wherein the reversed-phase adsorbent medium is selected from the group consisting of a hydrophobic resin, a magnesium-silicate gel, an alkyl-functionalized silica gel, a silica gel, and combinations thereof.

3. The method of claim 2, wherein the hydrophobic resin is CALBIOSORB™.

4. The method of claim 2, wherein the magnesium-silicate gel has the following chemical formula: MgO 3.75 SiO₂(x)H₂O.

5. The method of claim 2, wherein the alkyl-functionalized silica gel is an octyl-functionalized silica gel or an octadecyl-functionalized silica gel.

6. The method of claim 1, wherein the biological sample comprises a body fluid sample.

7. The method of claim 6, wherein the body fluid sample is selected from the group consisting of a blood specimen, a urine specimen, a spinal fluid specimen, and a peritoneal fluid specimen.

8. The method of claim 1, wherein the microorganism is a bacterium and the one or more inhibitors of microorganism growth is an antibiotic.

9. The method of claim 8, wherein the antibiotic is selected from the group consisting of a macrolide, a cephalosporin, a tetracycline, a fluoroquinolone, an aminoglycoside, a glycopeptide, and combinations thereof.

10. A medium for culturing a microorganism, the medium comprising a liquid nutrient medium and a reversed-phase adsorbent medium capable of selectively removing an inhibitor of microorganism growth, if present, from the medium.

11. The medium of claim 10, wherein the liquid nutrient medium is a hypertonic medium.

12. The medium of claim 10, wherein the reversed-phase adsorbent medium is selected from the group consisting of a hydrophobic resin, a magnesium silicate gel, a silica gel, an alkyl-functionalized silica gel, and combinations thereof.

13. The medium of claim 12, wherein the hydrophobic resin is CALBIOSORB™.

14. The medium of claim 12, wherein the magnesium silicate gel has the following chemical formula: MgO 3.75 SiO₂(x)H₂O.

15. The medium of claim 12, wherein the alkyl-functionalized silica gel is an octyl-functionalized silica gel or an octadecyl-functionalized silica gel.

16. The medium of claim 10, wherein the microorganism is a bacterium and the inhibitor of microorganism growth is an antibiotic.

17. The medium of claim 16, wherein the antibiotic is selected from the group consisting of a macrolide, a cephalosporin, a tetracycline, a fluoroquinolone, an aminoglycoside, a glycopeptides, and combinations thereof.

18. A method for growing and detecting in a body fluid sample an infecting microorganism, the method comprising:
(a) providing a culture growth medium comprising a liquid nutrient medium and a reversed-phase adsorbent medium capable of selectively removing an inhibitor of microorganism growth, if present, from the sample or the culture growth medium;
(b) inoculating the culture growth medium with the body fluid sample;
(c) culturing the inoculated culture growth medium under conditions sufficient to allow growth of the infecting microorganism; and
(d) detecting the growth of the infecting microorganism.

19. The method of claim 18, wherein the liquid nutrient medium is a hypertonic medium.

20. The method of claim 19, wherein the reversed-phase adsorbent material is selected from the group consisting of a hydrophobic resin, a magnesium silicate gel, a silica gel, an alkyl-functionalized silica gel, and combinations thereof.

21. The method of claim 20, wherein the hydrophobic resin is CALBIOSORB™.

22. The method of claim 20, wherein the magnesium silicate gel has the following chemical formula: MgO 3.75 SiO₂(x)H₂O.

23. The method of claim 18, wherein the infecting microorganism is a bacterium and the inhibitor of microorganism growth is an antibiotic.

24. The method of claim 18, wherein the detecting the growth of the infecting microorganism includes detecting metabolic byproducts produced by the infecting microorganism.

25. A culture growth receptacle for receiving a biological sample suspected of containing one or more inhibitors of microorganism growth, the receptacle comprising a culture growth medium and a reversed-phase adsorbent medium capable of selectively removing one or more inhibitors of microorganism growth, if present, in the sample or culture growth medium.
